(19) United States
(12) Patent Application Publication
(10) Pub. No.: US 2009/0124951 A1
(43) Pub. Date: May 14, 2009

(54) STAIN MASKING MATERIAL AND ARTICLES MADE THEREFROM

(75) Inventor: Ioannis V. Bletsos, Midlothian, VA (US)

Correspondence Address: E I DU PONT DE NEMOURS AND COMPANY;LEGAL PATENT RECORDS CENTER, BARLEY MILL PLAZA 25/1122B, 4417 LANCASTER PIKE, WILMINGTON, DE, 19805, US

(73) Assignee: E.I. DU PONT DE NEMOURS AND COMPANY

(21) Appl. No.: 12/268,464

(22) Filed: Nov. 11, 2008

Related U.S. Application Data
(60) Provisional application No. 61/003,036, filed on Nov. 14, 2007.

(51) Int. Cl. A61F 13/00; B01D 59/26; B65D 81/24

(52) U.S. Cl. 602/43; 96/154; 206/213.1

(57) ABSTRACT

Articles are provided which mask the appearance of stains. The articles utilize a metalized substrate formed from a substrate and a discontinuous metal layer deposited thereon, the metalized substrate having a gloss of at least about 2 gloss units when measured at an angle of illumination of 20°.

This application claims priority under 35 U.S.C. §119 from U.S. Provisional Application Ser. No. 61/003,036 (filed Nov. 9, 2007), the disclosure of which is incorporated by reference herein for all purposes as if fully set forth.

### BACKGROUND OF THE INVENTION

1. Field of the Invention

The present invention relates to materials suitable for use in various types of applications including packages which mask stains, such as stains produced by oil-containing substances. The invention furthermore relates to articles incorporating the materials.

2. Description of the Related Art

Known air permeable materials for use in packaging applications include paper, nonwoven sheet, porous films, perforated films and laminates thereof. However, it has been found that when such packaging materials are exposed to grease-, fat- or oil-containing substances, such materials become discolored by visible oil stains which are undesirable and may be unacceptable to consumers or users of the packages.

Air permeable materials are used in a variety of packaging applications where the materials may come in contact with oil, grease or fat (also referred to herein as simply “oil”), including, for example, as the walls of oxygen-scavenging packets used to regulate the exposure of oxygen-sensitive products within a package to oxygen in order to preserve the freshness, quality and “shelf-life” of the products. By limiting the exposure of oxygen-sensitive food products to oxygen within a package, the quality or freshness of food is maintained, spoilage is reduced, and the food shelf life can be extended. In the food packaging industry, oxygen exposure can be regulated by including together with the items within a package (e.g., cans, jars, plastic containers, bags, etc.) air permeable oxygen-scavenging sachets or packets (also referred to as “oxygen scavenging packets”) which contain a composition which scavenges the oxygen through oxidation reactions. The walls of such packets are air permeable so that oxygen can pass from outside the packet to the oxygen scavenging composition within the packet. Oxygen scavenging packets are used within packages of oily foods where they may become visibly stained.

Air permeable materials are also used in protective apparel applications in which an article of apparel is worn as personal protection during activities which result in staining of the apparel. Typically the apparel article is discarded solely because of the staining, while the functionality of the article has not been affected by the staining. It would be desirable to reduce the appearance of stains so that the useful life of the article can be extended.

It would be desirable to have a material suitable for applications including, but not limited to, the above applications which masks the appearance of stains, such as, but not limited to, oil stains, resulting from contact of the material with staining substances during use.

### SUMMARY OF THE INVENTION

One embodiment of the present invention is an oxygen-scavenging packet suitable for contact with oil-containing substances, wherein the packet comprises (a) a plurality of side walls having inner and outer surfaces and defining an enclosed space wherein each side wall comprises a metalized sheet comprising (i) a porous substrate having inner and outer surfaces, and (ii) a discontinuous metal layer deposited on the outer surface of the substrate; wherein the metalized sheet has a gloss at an angle of illumination of 20° greater than about 2 gloss units, a Gurley Hill porosity of less than about 20,000 seconds, and wherein the appearance of staining on the outer surfaces of the side walls is masked; and (b) an oxygen absorber within the enclosed space of the packet.

Another embodiment of the present invention is a package for enclosing an article comprising (a) a plurality of side walls having inner and outer surfaces and defining an enclosed space suitable for containing the article wherein each side wall comprises a metalized sheet comprising (i) a porous substrate having inner and outer surfaces, and (ii) a discontinuous metal layer deposited on the outer surface of the substrate; wherein the metalized sheet has a gloss at an angle of illumination of 20° greater than about 2 gloss units, and wherein the appearance of staining on the outer surfaces of the side walls is masked.

Another embodiment of the present invention is an article of apparel comprising a metalized substrate having inner and outer surfaces and a discontinuous metal layer deposited on the outer surface of the substrate; wherein the metalized substrate has a gloss at an angle of illumination of 20° greater than about 2 gloss units, a Frazier air permeability of at least about 0.1 cfm/ft² and a moisture vapor transmission rate in the range of 1000-3000 g/m²/24 hr, and wherein the appearance of staining on the outer surface of the metalized substrate is masked.

Another embodiment of the present invention is an article for dressing wounds comprising a metalized substrate having an inner surface and an outer surface and a discontinuous metal layer deposited on the outer surface of the substrate; wherein the metalized substrate has a gloss at an angle of illumination of 20° greater than about 2 gloss units, Frazier air permeability of at least about 0.1 cfm/ft², and wherein the appearance of staining on the outer surface of the metalized substrate is masked.

### DETAILED DESCRIPTION OF THE INVENTION

The invention is directed to the use of a metalized substrate including a porous substrate and a discontinuous metal layer deposited thereon, the metalized substrate having a gloss of at least about 2 gloss units when measured at an angle of illumination of 20°. The metalized substrate masks the appearance of stains, particularly stains caused by oil. The discontinuous metal layer is deposited on at least one surface of the porous substrate on which it is desirable to mask the appearance of stains.

Substrates for use as the porous substrate include woven fabrics, spunbond nonwovens, spunbonded-meltblown-spunbonded composite nonwovens, spunlaced nonwovens, nonwovens including submicron fibers such as electrospun or electroblown web, microporous film, paper, microperforated film and laminates thereof. Suitable woven fabrics, spunbond nonwovens, spunbonded-meltblown-spunbonded composite nonwovens can be formed from synthetic filaments such as polyester, polyamide and polyolefin filaments. Suitable microporous films can be formed from polymers such as, for example, polyester, polyamide, polycarbonate, vinyl, polyolefins and fluorinated polymers. Desirable properties of the substrate will vary depending on the embodiment of the invention.

Metals for use in the metal layer include aluminum, silver, copper, gold, tin, nickel, zinc, iron, and their alloys including aluminum bronze alloys, manganese bronze alloys, copper/nickel/zinc alloys, copper/nickel alloys, stainless steel, and aluminum/magnesium alloys. The metal layer can have a thickness between about 15 nanometers and 200 nanometers. Alternatively, the metal layer can be formed from a plurality of superimposed metal layers wherein each metal layer has the same or different metal composition relative to the other metal layers. The metal layer is discontinuous as the metal layer does not completely cover the pores of the substrate.

The discontinuous metal layer can be left uncoated or coated as desired for the application. According to one embodiment of the invention, a package is provided which masks the appearance of stains. In packaging applications in which the package will come in contact with food, the metal layer can be coated by a thin layer of an FDA-approved polymer such as a polyacrylate which is advantageously also printable. The metalization of the porous substrate and subsequent coating can economically be carried out in a single pass using a vapor deposition process as disclosed in co-pending U.S. patent application having Ser. No. 10/924,218, filed on Aug. 23, 2004, incorporated by reference herein in its entirety. The metallization can be carried out by thermal evaporation, sputtering or other metal deposition technologies known in the art. The metal layer may be passivated and the coating on top of it may occur in a different step sometime after metallization. If the surface of the polyacrylate coating is printed on, a second coating can be applied over the ink to prevent the printing ink from contacting the food and also from wearing or fading.

Packages can be formed from a plurality of side walls of the packaging material of the invention defining an enclosed space suitable for containing an article or a plurality of articles. Packages made according to the invention can be formed from material which is air permeable and which regulates the exposure of items or materials to oxygen or moisture. Packages made from such air permeable materials are referred to as “intelligent” or “active” packaging systems. The package can include a heat sealable layer located on surfaces where the side walls are adhered to each other, e.g., a layer of low density polyethylene, polypropylene or copolymers of ethylene and vinyl acetate. The amount of the heat sealable layer can also be used to regulate the air permeability of the package.

According to another embodiment of the invention, an oxygen scavenging packet is provided which masks the appearance of stains. The oxygen scavenging packet has a plurality of side walls defining an enclosed space to contain an oxygen absorber composition. The oxygen scavenging packet can utilize a metalized substrate in each of the side walls of the packet or in fewer than all of side walls of the packet and an air impermeable material can be used in the remainder of the side walls. The air permeable material used in oxygen scavenging packets of the invention has an air permeability of less than 20,000 Gurley seconds. This permeability regulates the rate of oxygen that can be absorbed by the absorber within the packet. This determines the useful life of the packet which in turn influences the shelf life of the oxygen-sensitive contents the oxygen scavenging packet is intended to protect. Oxygen scavenging packets are formed from side wall materials such as the packaging material of the invention, using any known process for forming the packets including “form-fill-seal” processes in which tubes or other enclosures of the packaging material are formed continuously while the packaging material is unwound from a roll. Once the tube is formed, cross machine direction seals are formed in the tube creating three sided pouches which are then filled with the contents of the packet. The packets are finally sealed completely. Oxygen absorbers for use in the oxygen scavenging packet of the invention include any known oxygen absorber compositions, such as those disclosed in U.S. Pat. No. 5,241,149.

According to another embodiment of the invention, articles of apparel, bedding, diapers and sanitary articles which mask the appearance of stains can advantageously be formed from a metalized porous substrate. For instance, medical garments and protective apparel items formed from substrates having a discontinuous layer of metal deposited on the outer surface mask the appearance of oil stains, blood stains, etc. For such uses, the substrate is advantageously selected from woven fabrics, spunbond nonwovens, spunlaced nonwovens, nonwovens including submicron fibers, spunbonded-meltblown-spunbonded composite nonwovens, and laminates thereof. The substrate has a Frazier air permeability from about 0.1 to more than about 100 cfm/ft² and a moisture vapor transmission rate in the range of 1000-3000 g/m²/24 hr.

According to another embodiment of the invention, wound dressing materials can be formed from a metalized substrate in which a discontinuous layer of metal is deposited onto one surface of a porous substrate (“outer surface”). When the non-metalized surface of the substrate (“inner surface”) is placed directly over a wound, stains caused by blood and other fluids including topical treatments applied to the wound are not visible on the outer surface of the wound dressing. The wound dressing materials of the invention can also be used in adhesive bandages. For this use, the substrate is advantageously selected from woven fabrics, spunbond nonwovens, spunlaced nonwovens, nonwovens including submicron fibers, spunbonded-meltblown-spunbonded composite nonwovens, and laminates thereof. The substrate has a Frazier air permeability from about 0.1 to more than about 100 cfm/ft² and a moisture vapor transmission rate in the range of 1000-3000 g/m²/24 hr.

According to another embodiment of the invention, stationery and graphics items which mask stains are provided, including, but not limited to, envelopes, shipping pouches, substrates for graphic images, maps, business cards, banners, etc. For this use, suitable substrates advantageously have a tensile strength of at least about 20 lb/in according to ASTM D5035, even between about 20 lb/in and about 72 lb/in, and an elongation of at least about 14% according to ASTM D5035, even between about 14% and about 29%. Suitable substrates advantageously include spunbond nonwovens, film, paper, and laminates thereof.

According to yet another embodiment of the invention, sheet which masks stains are also provided which are useful in agricultural sheet applications such as mulches and sheet used to reflect solar radiation and facilitate the drying of raisins. For these uses, suitable substrates advantageously have a tensile strength of at least about 20 lb/in according to ASTM D5035, even between about 20 lb/in and about 72 lb/in, and an elongation of at least about 14% according to ASTM D5035, even between about 14% and about 29%, reflectivity in the visible and infrared spectra, and high UV durability. Suitable substrates for such uses advantageously include woven fabrics, spunbond nonwovens, film, paper and laminates thereof.

### Test Methods

Gloss was measured according to ASTM D2457-03 at an angle of illumination of 200.

### EXAMPLES

Two rolls of high density polyethylene spunbond nonwoven sheet commercially available from E. I. du Pont de Nemours & Co. (Wilmington, Del.) under the trade name DuPont™ Tyvek® (style 1560B) were treated with an 80% argon/20% nitrogen plasma in vacuum and were subsequently metalized with a discontinuous layer of aluminum using resistive evaporation. Both steps were done in a single pass. One roll was metalized on one side only (designated “single metal”) and the other roll was metalized on both sides (designated “double metal”). Neither roll was coated after metallization. The resulting “single metal” roll had a shiny metalized side and a white side. The “double metal” roll had two shiny metalized sides that could be distinguished from each other by the original surface patterns of the Tyvek® sheet, in which one side was relatively smooth and the other side was relatively rough.

Samples of the “single metal” and the “double metal” Tyvek® rolls were folded as follows:

### Example 1

### Single Metal Sample, Metalized Side Folded onto Metalized Side

### Example 2

### Single Metal Sample, White Side Folded onto White Side

### Example 3

### Double Metal Sample, Rough Metalized Side Folded onto Rough Metalized Side

Three pieces of pepperoni of equal thickness were placed side by side within the fold of each of the three samples as described above and completely covered on each side by the samples. In Example 1 the metalized surface contacts the pepperoni slices; in Example 2 the white, non-metalized surface contacts the pepperoni slices; and in Example 3 the rough, metalized surface contacts the pepperoni slices. A 5 lb (2.3 kg) weight was placed on top of each folded sample with the pepperoni pieces inside the fold to press oil from the pepperoni slices into direct contact with the surrounding samples. The weight was left in place on each sample for approximately 10 hours. The weight was removed, the samples were unfolded and the pieces of pepperoni were removed to observe the stain on the metalized Tyvek® samples.

In the single-sided metalized samples, regardless of whether the pepperoni was in contact with the white (non-metalized) or the metalized side, the white side showed a circular oily stain surrounded by a reddish “halo.” No oil stain or reddish halo was observed on the metalized side, regardless of whether the pepperoni was in contact with the white or metalized side. Similarly, in the double-sided metalized samples, regardless of whether the pepperoni was in contact with the smooth or rough side, no oily stain or reddish halo was observed on either side.

The gloss at an angle of illumination of 20° was measured for each side of each sample, both outside the circular area (“outside”) which was covered by the weight and inside the circular area (“inside”), and the results are listed in Table 1. Each gloss measurement listed is the average of the gloss of the surface above the pepperoni slices and the gloss of the surface below pepperoni slices. The percent difference in gloss was calculated as the percent difference between the gloss outside the circular area and the gloss inside the circular area relative to the gloss unaffected by the stain, i.e., outside the circular area.

Control sample gloss measurements were also taken on clean samples having no contact with the pepperoni. Control 1 was a clean “single metal” sample. Control 2 was a clean “double metal” sample.

TABLE 1

| | | | % |
|---|---|---|---|
| | | | Difference |
| | | Gloss | in Gloss |
| Control 1 | Metalized surface | 5.5 | |
| | Non-metalized surface | 1.5 | |
| Example 1 | Outside, metalized surface | 4.9 | 14 |
| | Inside, metalized surface | 4.2 | |
| | Outside, non-metalized surface | 1.2 | 17 |
| | Inside, non-metalized surface | 1.0 | |
| Example 2 | Outside, metalized surface | 5.4 | 8.4 |
| | Inside, metalized surface | 4.9 | |
| | Outside, non-metalized surface | 1.6 | 35 |
| | Inside, non-metalized surface | 1.0 | |
| Control 2 | Rough metalized surface | 3.1 | |
| | Smooth metalized surface | 5.4 | |
| Example 3 | Outside, rough metalized surface | 3.0 | 15 |
| | Inside, rough metalized surface | 2.5 | |
| | Outside, smooth metalized surface | 5.4 | 6.5 |
| | Inside, smooth metalized surface | 5.0 | |

The metalized surfaces have higher gloss than the non-metalized white sides and they hide the appearance of the oil stain. In both the metalized sides and the white sides of the above samples, the gloss is reduced inside the circular area which was covered by the weight, however the reduction of gloss by the oily stain is significantly greater on the white, non-metalized surface than on the metalized surfaces of the Examples.

What is claimed is:

1. An oxygen-scavenging packet suitable for contact with oil-containing substances, wherein the packet comprises:
(a) a plurality of side walls having inner and outer surfaces and defining an enclosed space wherein each side wall comprises a metalized sheet comprising:
(i) a porous substrate having inner and outer surfaces, and
(ii) a discontinuous metal layer deposited on the outer surface of the substrate;
wherein the metalized sheet has a gloss at an angle of illumination of 20° greater than about 2 gloss units, a Gurley Hill porosity of less than about 20,000 seconds, and wherein the appearance of staining on the outer surfaces of the side walls is masked; and
(b) an oxygen absorber within the enclosed space of the packet.

2. The oxygen-scavenging packet of claim 1 wherein the substrate is selected from the group consisting of woven fabrics, spunbonded nonwovens, spunlaced nonwovens, nonwovens including submicron fibers, spunbonded-meltblown-spunbonded composite nonwovens, microporous film, paper, and laminates thereof.

3. The oxygen-scavenging packet of claim 1 wherein the metalized sheet further comprises a layer comprising electron beam curable ink disposed on the metal layer.

4. The oxygen-scavenging packet of claim 1 wherein the metalized sheet further comprises a polymer coating layer.

5. The oxygen-scavenging packet of claim 1 wherein the metal layer comprises the metal selected from the group consisting of aluminum, silver, copper, gold, tin, nickel, zinc, iron, and alloys thereof.

6. A package for enclosing an article comprising:
(a) a plurality of side walls having inner and outer surfaces and defining an enclosed space suitable for containing the article wherein each side wall comprises a metalized sheet comprising:
(i) a porous substrate having inner and outer surfaces, and
(ii) a discontinuous metal layer deposited on the outer surface of the substrate;
wherein the metalized sheet has a gloss at an angle of illumination of 20° greater than about 2 gloss units, and wherein the appearance of staining on the outer surfaces of the side walls is masked.

7. The package of claim 6 further comprising a heat sealing layer disposed between the side walls wherein the heat sealing layer comprises a polyolefin polymer or copolymer.

8. An article of apparel comprising:
a metalized substrate having inner and outer surfaces and a discontinuous metal layer deposited on the outer surface of the substrate;
wherein the metalized substrate has a gloss at an angle of illumination of 20° greater than about 2 gloss units, a Frazier air permeability of at least about 0.1 cfm/ft² and a moisture vapor transmission rate in the range of 1000-3000 g/m²/24 hr, and wherein the appearance of staining on the outer surface of the metalized substrate is masked.

9. An article for dressing wounds comprising a metalized substrate having an inner surface and an outer surface and a discontinuous metal layer deposited on the outer surface of the substrate;
wherein the metalized substrate has a gloss at an angle of illumination of 20° greater than about 2 gloss units, Frazier air permeability of at least about 0.1 cfm/ft², and wherein the appearance of staining on the outer surface of the metalized substrate is masked.
